(19) United States
(12) Patent Application Publication
(10) Pub. No.: US 2026/0328677 A1
(43) Pub. Date: Oct. 22, 2026

(54) PHOTOCHEMICAL FOOTPRINTING OF TARGETED PROTEINS FROM MAMMALIAN BODILY FLUIDS

(71) Applicants: University of Mississippi, University, MS (US); The Regents of the University of California, Oakland, CA (US)

(72) Inventors: Joshua Shane SHARP, University, MS (US); Darrienne Myia MARTIN, University, MS (US); Lisa Michelle JONES, La Mesa, CA (US)

(21) Appl. No.: 19/484,006

(22) PCT Filed: May 14, 2024
(86) PCT No.: PCT/US2024/029220

§ 371 Date: Nov. 13, 2025

Related U.S. Application Data
(60) Provisional application No. 63/502,133, filed on May 15, 2023.

(51) Int. Cl. G01N 21/27; G01N 1/40

(52) U.S. Cl. CPC G01N 21/272; G01N 1/4044

(57) ABSTRACT

In one aspect, the disclosure relates to methods and systems for evaluating the higher-order structure of a target biomolecule, such as, for example, a protein, within a biological fluid. The biological fluid can be isolated from a mammal or another multicellular host and subjected to the method without further processing steps. In an aspect, the biological fluid is labeled to produce a labeled target biomolecule, which can then be analyzed using LC-MS/MS or another technique to produce a labeled target molecule profile, which can be compared to an LC-MS/MS profile of the unlabeled target biomolecule, wherein m/z and retention times of the labeled target biomolecule or degradation products thereof can be compared to those for unlabeled target biomolecules in order to elucidate details about the structure of the target biomolecule including locations of solvent-accessible residues, conformation changes in a particular extracellular environment, drug-target biomolecule interactions, and the like.

### CROSS REFERENCE TO RELATED APPLICATIONS

This application claims the benefit of U.S. Provisional Application No. 63/502,133 filed on May 15, 2023, which is incorporated herein by reference in its entirety.

### STATEMENT REGARDING FEDERALLY SPONSORED RESEARCH OR DEVELOPMENT

This invention was made with government support under grant number R01GM127267, awarded by the National Institutes of Health. The government has certain rights in the invention.

### BACKGROUND

Structural biology plays a central role in modern molecular biosciences, enabling a greater understanding of molecular mechanisms of biomolecular action and enabling the rational design of molecules to modulate these interactions. However, atomic resolution experimental technologies have important limitations that prevent analyses of certain important macromolecular structural systems. Dynamic systems are especially challenging for both experimental and structure prediction methods. Systems such as post-translationally modified proteins, protein-carbohydrate complexes and protein oligomerization/aggregation products are notoriously difficult to structurally characterize. Similarly, structural characterization of proteins in vivo or within a similar matrix is highly challenging, but crucial for understanding a molecular system in its natural biological context. Technologies to address these systems need to be able to handle the dynamic and diverse array of proteoforms and conformers, as well as dynamic oligomerization states.

This gap in knowledge has largely left experimental space for biophysical characterization that either aids in assembly of well-defined substructures or that results in biophysical information without a rigorous molecular model, including various modes of spectroscopy and, more recently, multiple varieties of structural mass spectrometry. The data generated by these technologies are valuable, but usually cannot generate experimental molecular models in the absence of an atomic structure. The development of a biophysical tool that has flexible analysis characteristics, high sensitivity to deal with small sample sizes, requires relatively little analysis time, and provides data that are readily compatible with the development and/or evaluation of high quality computational molecular models would offer pathways to the development of experimentally-based molecular models of systems currently unrepresented in the database.

Despite advances in structural biology research, there is still a scarcity of effective methods for probing protein structure and protein interactions with pharmaceuticals and their metabolites in living mammals in real time. A major unmet need in structural pharmacology is the ability to probe how protein pharmaceutical structure and complexes form in the blood at various times during the drug's lifetime in mammalian blood. An ideal method for doing so would alleviate the major issues of traditional footprinting experiments: namely, the induction of label-induced structural artifacts, the limited range of amino acids that can be probed in a single experiment, and the lack of quantitative correlation between solvent accessible surface area (SASA) and apparent reaction rate due to the influence of other factors. In one aspect, it would be desirable to measure SASA for multiple amino acids in a single experiment so as to determine protein structure of unknown proteins. An ideal method would further allow for study of structural impacts of a drug on a drug target as well as to identify off-target biomolecules. Such a method could measure direct structural changes due to drug binding as well as more complex changes in drug target interactions with other components of the biofluid, even if the interaction partners are unknown. The method would further enable understanding of the molecular foundation behind adverse drug events, off-target drug interactions, and the like. These needs and other needs are satisfied by the present disclosure.

### SUMMARY

In accordance with the purpose(s) of the present disclosure, as embodied and broadly described herein, the disclosure, in one aspect, relates to methods and systems for evaluating the higher-order structure of a target biomolecule, such as, for example, a protein, within a biological fluid. The biological fluid can be isolated from a mammal or another multicellular host and subjected to the method without further processing steps. In an aspect, the biological fluid is labeled to produce a labeled target biomolecule, which can then be analyzed using LC-MS/MS or another technique to produce a labeled target molecule profile, which can be compared to an LC-MS/MS profile of the unlabeled target biomolecule, wherein m/z and retention times of the labeled target biomolecule or degradation products thereof can be compared to those for unlabeled target biomolecules in order to elucidate details about the structure of the target biomolecule including locations of solvent-accessible residues, conformation changes in a particular extracellular environment, drug-target biomolecule interactions, drug-protein interactions to measure off-target and anti-drug antibody interactions, and the like.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows a schematic of an exemplary whole blood fluidics system useful in the methods described herein. FIG. 1B shows a second exemplary whole blood fluidics system including a semi-automated switching valve.

FIG. 2A shows a process for oxidative modification oof the side chains of amino acids as part of an initial step for hydroxyl radical protein footprinting (HRPF). FIG. 2B shows that changes in protein conformation based on solvent availability as measured by irreversible hydroxyl radical labeling can be calculated.

FIG. 3 shows a schematic of fast photochemical oxidation of proteins (FPOP).

FIGS. 4A-4C show a schematic of an exemplary process for analyzing proteins in whole blood. A similar process can be used for both ex vivo and in vitro experiments.

FIGS. 5A-5C show blood catalase activity measured by foam assay. Representative images of results shown: (FIG. 5A), 500 μL of blood was spiked with 25 μL of: 100 mM sodium phosphate pH 7.4, final 5 mM HA, final 25 mM HA, final HA 100 mM, and final HA 142 mM. (FIG. 5B) 920 μL of blood spiked with either 80 μL of 30% H₂O₂ or 80 μL of 2.5M Na₂S₂O. Foam height in test tube (FIG. 5C) after 30 minute incubation at 5:1 v/v blood to inhibitor at room temperature. Note that TSC is not soluble at 200 mM. Error bars represent one standard deviation of triplicate measurement.

FIGS. 6A-6C show adenine dosimetry response to (FIG. 6A) changing UV lamp voltage; (FIG. 6B) changing sodium persulfate concentrations; or (FIG. 6C) changing radical scavenger (sucrose) concentrations. All three methods change the effective sulfate radical dose. Experiments were conducted in vitro.

FIGS. 7A-7B show the number of oxidative events per peptide labeled by FOX footprinting for (FIG. 7A) purified mouse albumin labeled in vitro and (FIG. 7B) mouse albumin labeled ex vivo in mouse whole blood. Error bars represent one standard deviation from a triplicate measurement. In vitro mouse albumin displayed substantial oxidation from the purification and storage process; reported values are corrected for pre-existing oxidation.

FIGS. 8A-8B show analysis of mixing in the blood RPF flow system. FIG. 8A: Deconvoluted images of the red dye channel at 2 cm from tee (top) and 4 cm from tee (bottom). The red dye enters the tee on the side of the capillary near the top of the image; a color gradient can be observed at 2 cm that is gone at 4 cm. FIG. 8B: Histogram analysis of red channel color density across the capillary at 2, 3 and 4 cm. A distribution of color intensity can be clearly seen to 2 cm that collapses to homogeneity at 4 cm.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are a system and method for ex vivo analysis of a defined number of biomolecules of interest. In an aspect, the system and/or method are used to develop a targeted LC-MS/MS method for high coverage analysis of the biomolecules of interest from a highly complex matrix. In some aspects, specific degradation of large biomolecules (e.g. the use of a protease to digest a protein into peptides) can be used to simplify analysis when appropriate. In a further aspect, the mass-to-charge ratio (m/z) and retention time of potential peptide covalent labeling products can be modeled based on the known chemistry of the labeling reagents. Further in this aspect, a modeled retention time shift window can be applied to the measured retention time of the unmodified biomolecule or biomolecule degradation product.

In a further aspect, using the disclosed method, structural changes to a targeted drug, such as, for example, adalimumab, in stabilized mammalian whole blood can be observed. In another aspect, the blood can be tested for hemolysis to ensure integrity during labeling.

In one aspect, in the disclosed method, isolated biofluids (chemically stabilized when necessary for handling) are passed through a fluidic channel with a sheath flow containing a photoactivatable labeling precursor. Further in this aspect, the sheath flow both provides the precursor and prevents aggregation of any cellular components. In a still further aspect, the biofluid is passed through the path of an ultrafast pulsed light source, focused on the lumen of the fluidic channel and timed to illuminate each volume of the biofluid with a controlled number of illumination events. In one aspect, the illumination photoactivates the covalent labeling precursor, labeling biomolecules within the biofluid. In another aspect, the reaction is collected, quenching any long-lived products if necessary. In yet another aspect, the resulting complex biofluid containing the protein target(s) is processed for targeted LC-MS/MS analysis to quantify the amount of photochemical labeling using the method established in the first paragraph. In one aspect, the accessibility of a specific region of the targeted biomolecule(s) of interest is related to the measured amount of labelling that occurs at that region.

Also disclosed herein are methods for performing HR-HRPF in stabilized whole blood, using isoforms of adalimumab within dosed mice as a test system. In an aspect, adalimumab and TNFα can be labeled in both stabilized mouse whole blood and in plasma, for both WT mice and for ΔTNFα mice. In a further aspect, protein depletion and/or enrichment can be used if necessary to improve sequence coverage of the targeted proteins, and multiplexing strategies can be used to improve the reproducibility of sequence coverage and reliability of quantitation. In a further aspect, the known epitope:paratope interface can be used to validate the results. In one aspect, adalimumab versions engineered into the four IgG subclasses can be tested to look for differences in interactions of different IgG subclasses with whole blood components. In a further aspect, these results will be compared with known differences in interactions between the subclasses and components in the immune response system. In any of these aspects, it is understood that adalimumab operates as a non-limiting test system for the disclosed methods.

In an aspect, a combination of ultra-fast covalent biomolecular covalent surface mapping (or “footprinting”) can be combined with high resolution LC-MS/MS to measure biomolecular solvent accessible surfaces, (sometimes referred to in the field as biomolecular topography).

In one aspect, disclosed herein is a method for evaluating a higher-order structure of a target biomolecule within a biological fluid, the method including at least the steps of:
(a) isolating a sample including a biological fluid from a multicellular host;
(b) subjecting the sample comprising the biological fluid to at least one labeling condition to produce a labeled target biomolecule;
(c) analyzing the labeled target biomolecule using targeted spectrometry to produce a labeled target molecule profile for the target biomolecule from a complex background; and
(d) comparing the labeled molecule profile to a reference molecule profile of an unmodified target biomolecule;
wherein a m/z (mass to charge) ratio and retention time of a labeled target biomolecule or degradation product thereof is modeled based on known chemical behavior of the at least one labeling condition; and
wherein a modeled retention time shift window is applied to a measured retention time of the unmodified target biomolecule or a degradation product thereof.

In some aspects, in the disclosed method the reference molecule profile can be a labeling profile generated from the isolated, purified, or enriched target biomolecule from an in vitro sample, and wherein the reference molecule profile is used to model the labeled target molecule profile in the biological fluid in step (c). In a further aspect, the biological fluid can be whole blood, serum, plasma, urine, cerebrospinal fluid (CSF), another biological fluid, or any combination thereof. In any of these aspects, the biological fluid does not require purification before performing the method. In another aspect, the multicellular host can be a mammal such as, for example, a human, cat, dog, goat, sheep, rabbit, cattle, swine, hamster, rat, mouse, guinea pig, horse, ferret, camel, llama, or non-human primate.

In an aspect, the at least one labeling condition can be oxidizing condition, CF₃ radicals, SO₄ radicals, CO₃ radicals, I radicals, carbenes, aryl azides, benzophenones, or any combination thereof. When the at least one labeling condition is an oxidizing condition, the biological fluid can be contacted with at least one oxidizing agent such as, for example, a peroxide and irradiating the biological fluid and the at least one oxidizing agent. In another aspect, if the oxidizing agent is a peroxide, the peroxide can be hydrogen peroxide, sodium persulfate, or another peroxide, including any symmetric or asymmetric organic peroxide, mixed hydroperoxide, or the like. In an aspect, different peroxides can be used in order to change the rate of oxidation, to avoid inactivation by other compounds in the biological sample (e.g. catalase with hydrogen peroxide), or both. In an aspect, the at least one peroxide can be present at from about 10 mM to about 1000 mM, from about 10 mM to about 500 mM, from about 50 mM to about 175 mM, from about 75 mM to about 150 mM, or at about 100 mM. In some aspects, the sample containing the biological fluid and the at least one peroxide further includes a radical scavenger, such as, for example, adenine. In a further aspect, the adenine can be present at about from about 0.1 mM to about 10 mM, or from about 0.5 mM to about 5 mM, or at about 1 mM. In still another aspect, the sample containing the biological fluid and the at least one peroxide further includes a buffer such as, for example, sodium phosphate or another buffer that maintains the sample at a pH of from about 5 to about 9, or from about 6 to about 8, or from about 6.5 to about 7.5, or of about 7.2. In one aspect, adenine is especially useful as a radical dosimeter (i.e., a scavenger that has a conveniently measurable changes in properties upon reaction). Other radical scavengers are also contemplated and should be considered disclosed.

In an alternative aspect, the biological fluid can be contacted with a carbene-generating reagent, a trifluoromethyl radical-generating reagent, a benzophenone, or any combination thereof. In one aspect, the irradiation is accomplished using UV light having a wavelength of between about 200 nm and about 360 nm, or between about 225 and about 300 nm, or using 248 nm light. In some aspects, irradiating the biological fluid and the at least one peroxide includes one or more short irradiation events, wherein a short irradiation event includes irradiating the biological fluid and the at least one peroxide for a period of time under one second.

In any of these aspects, the sample can be quenched prior to further analysis prior to analyzing the sample via LC-MS/MS or another mass spectrometry technique. In one aspect, quenching includes the step of contacting the sample containing the biological fluid with a quenching solution including at least an imidazole or quinone and a thioether. In one aspect, the quenching solution includes imidazole, dimethylthiourea (DMTU), and methionine amide (Met-NH₂), such as a solution having 200 mM imidazole, 200 mM DMTU, and 70 mM Met-NH₂. In an aspect, and without wishing to be bound by theory, for persulfate labeling, useful compounds include a substance with an imidazole or quinone structure and a thioether. A thiourea or similar compound can be used to supplement the thioether, since thioethers are expensive and less shelf stable, allowing use of less of the thioether. In one aspect, the quenching solution can contain a buffer such as, for example, sodium phosphate or another buffer that maintains pH of from about 2.5 to about 10, or from about 5 to about 9, or rom about 6 to about 8, or at about 7.2. In one aspect, any buffer compatible with the biological sample and poorly reactive to labeling molecules can be used.

In one aspect, the method further includes contacting the sample containing the biological fluid with a digestive enzyme such as, for example, a protease, a nuclease, a lyase, a glycosidase, or any combination thereof. In another aspect, the digestive enzyme can be trypsin, chymotrypsin, GluC, LysC, PNGaseF, or any combination thereof.

In some aspects, biological fluids can contain enzymes such as, for example, catalase, that degrade labels on the target biomolecules. In a further aspect, these enzymes can be inhibited or degraded prior to the addition of the oxidizing agent. In one aspect, when the biological sample includes whole blood, the method further includes contacting the biological sample with a peroxidase inhibitor, a catalase inhibitor, or both. In one aspect, the catalase inhibitor can be a compound such as, for example, hydroxylamine, 3-amino-1,2,4-triazole (3AT), aminoguanidine, thiosemicarbazide (TSC), or any combination thereof. In any of these aspects, the sample including the biological fluid may not be purified prior to performing step (b) of the method. In an aspect, the catalase inhibitor can be used under conditions that maintain the general physiological state of the biological fluid.

In any of these aspects, the target biomolecule can be a protein or peptide, including, but not limited to, protein, a peptide, a carbohydrate, a nucleic acid, or any combination thereof. In another aspect, the target biomolecule can be a biomolecular drug (also known as a “biologic”). In still another aspect, the biomolecular drug can be a therapeutic monoclonal antibody or a protein-based pharmaceutical. In still another aspect, the target biomolecule can be a target molecule for a small molecule drug. In an aspect, the target biomolecule can be albumin or myoglobin. In another aspect, the target biomolecule can be a carbohydrate or a nucleic acid. In still another aspect, the target molecule can be a combination of two or more biomolecule types including, but not limited to, a glycosylated protein, a proteoglycan, or the like.

In any of these aspects, the method can be conducted in a flow system wherein the sample comprising the biological fluid is flowed through the flow system, wherein the method is conducted sequentially on one or more subvolumes of fluid moving through the flow system.

In one aspect, disclosed herein is a system for evaluating a higher-order structure of a target biomolecule within a biological fluid, the system including at least the following:
(a) a fluidic channel for receiving an isolated biological fluid containing a target biomolecule;
(b) a mixing portion for admixing one or more labeling reagents with the isolated biological fluid to produce a labeled target biomolecule, wherein the mixing portion is coupled to the fluidic channel for receiving the isolated biological fluid; and
(c) an automated system for receiving the labeled target biomolecule from the mixing portion.

In another aspect, the system can also include an irradiation module for irradiating the one or more labeling reagents and the isolated biological fluid, a quenching module for quenching the sample after labeling, and/or a second mixing portion for admixing one or more buffers with the isolated biological fluid.

In some aspects, the disclosed system further includes an in-line spectrophotometry module for analysis of biological fluid integrity, the spectrophotometry module being located between the fluidic channel for receiving the isolated biological fluid and the mixing portion. In a further aspect, the in-line spectrophotometry module is configured to collect UV/vis spectra, fluorescence spectra, light scattering measurements, or any combination thereof.

In another aspect, the system further includes a mass spectrometry module for identifying the target biomolecule and/or fragments or fragmentation products thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a buffer,” “a protein,” or “an antibody,” include, but are not limited to, mixtures or combinations of two or more such buffers, proteins, or antibodies, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an oxidant refers to an amount that is sufficient to achieve the desired improvement in the property modulated by the formulation component, e.g. achieving the desired level of protein oxidation. The specific level in terms of wt % in a composition required as an effective amount will depend upon a variety of factors including the identity of the oxidant (e.g. H₂O₂), concentration of the oxidant, target protein, and medical purpose for which protein structure is being monitored.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for evaluating a higher-order structure of a target biomolecule within a biological fluid, the method comprising:
(a) isolating a sample comprising a biological fluid from a multicellular host;
(b) subjecting the sample comprising the biological fluid to at least one labeling condition to produce a labeled target biomolecule;
(c) analyzing the labeled target biomolecule using targeted spectrometry to produce a labeled target molecule profile for the target biomolecule from a complex background; and
(d) comparing the labeled molecule profile to a reference molecule profile of an unmodified target biomolecule;
wherein a m/z (mass to charge) ratio and retention time of a labeled target biomolecule or degradation product thereof is modeled based on known chemical behavior of the at least one labeling condition; and
wherein a modeled retention time shift window is applied to a measured retention time of the unmodified target biomolecule or a degradation product thereof.

Aspect 2. The method of aspect 1, wherein the reference molecule profile comprises a labeling profile generated from the isolated, purified, or enriched target biomolecule from an in vitro sample, and wherein the reference molecule profile is used to model the labeled target molecule profile in the biological fluid in step (c).

Aspect 3. The method of aspect 1, wherein the biological fluid comprises whole blood, serum, plasma, urine, cerebrospinal fluid (CSF), or any combination thereof.

Aspect 4. The method of aspect 1, wherein the multicellular host is a mammal.

Aspect 5. The method of aspect 4, wherein the mammal is a human, cat, dog, goat, sheep, rabbit, cattle, swine, hamster, rat, mouse, guinea pig, horse, ferret, camel, llama, or non-human primate.

Aspect 6. The method of aspect 1, wherein the at least one labeling condition comprises an oxidizing condition, CF₃ radicals, SO₄ radicals, CO₃ radicals, I radicals, carbenes, aryl azides, benzophenones, or any combination thereof.

Aspect 7. The method of aspect 6, wherein subjecting the sample comprising the biological fluid to the oxidizing condition comprises contacting the biological fluid with at least one peroxide and irradiating the biological fluid and the at least one peroxide.

Aspect 8. The method of aspect 7, wherein the at least one peroxide comprises hydrogen peroxide (H₂O₂), sodium persulfate (Na₂S₂O₈), a mixed hydroperoxide, or any combination thereof.

Aspect 9. The method of aspect 8, wherein the at least one peroxide is sodium persulfate.

Aspect 10. The method of aspect 9, wherein the at least one peroxide is present at from about 10 mM to about 1000 mM.

Aspect 11. The method of aspect 7, wherein the at least one peroxide is present at from about 10 mM to about 500 mM.

Aspect 12. The method of aspect 7, wherein the sample comprising the biological fluid and the at least one peroxide further comprises a radical scavenger.

Aspect 13. The method of aspect 12, wherein the radical scavenger comprises adenine.

Aspect 14. The method of aspect 13, wherein the adenine is present at about 0.1 mM to about 10 mM.

Aspect 15. The method of aspect 7, wherein the sample comprising the biological fluid and the at least one peroxide further comprises a buffer.

Aspect 16. The method of aspect 15, wherein the buffer has a pH of from about 5 to about 9.

Aspect 17. The method of aspect 15, wherein the buffer comprises sodium phosphate.

Aspect 18. The method of aspect 7, wherein the biological fluid and the at least one peroxide are irradiated with a UV lamp having a wavelength greater than or equal to about 200 nm.

Aspect 19. The method of aspect 7, wherein irradiating the biological fluid and the at least one peroxide comprises one or more short irradiation events, wherein a short irradiation event comprises irradiating the biological fluid and the at least one peroxide for a period of time under one second.

Aspect 20. The method of aspect 1, further comprising quenching the sample comprising the biological fluid prior to performing step (c).

Aspect 21. The method of aspect 20, wherein quenching comprises contacting the sample comprising the biological fluid with a quenching solution comprising an imidazole or quinone and a thioether.

Aspect 22. The method of aspect 21, wherein the quenching solution consists of imidazole, dimethylthiourea (DMTU), and methionine amide (Met-NH₂).

Aspect 23. The method of aspect 21, wherein the quenching solution further comprises a buffer.

Aspect 24. The method of aspect 23, wherein the buffer has a pH of from about 2.5 to about 10.

Aspect 25. The method of aspect 23, wherein the buffer comprises sodium phosphate.

Aspect 26. The method of aspect 1, wherein the method further comprises contacting the sample comprising the biological fluid with a digestive enzyme.

Aspect 27. The method of aspect 26, wherein the digestive enzyme comprises a protease, a nuclease, a lyase, a glycosidase, or any combination thereof.

Aspect 28. The method of aspect 27, wherein the digestive enzyme comprises trypsin, chymotrypsin, GluC, LysC, PNGaseF, or any combination thereof.

Aspect 29. The method of aspect 3, wherein the biological sample comprises whole blood, and wherein the method further comprises contacting the biological sample with a catalase inhibitor, a peroxidase inhibitor, or any combination thereof.

Aspect 30. The method of aspect 29, wherein the catalase inhibitor comprises hydroxylamine, 3-amino-1,2,4-triazole (3AT), aminoguanidine, thiosemicarbazide (TSC), or any combination thereof.

Aspect 31. The method of aspect 1, wherein the sample comprising the biological fluid is not purified prior to performing step (b) of the method.

Aspect 32. The method of aspect 1, wherein the target biomolecule comprises a protein, a peptide, a carbohydrate, a nucleic acid, or any combination thereof.

Aspect 33. The method of aspect 1, wherein the target biomolecule comprises a biomolecular drug.

Aspect 34. The method of aspect 33, wherein the biomolecular drug comprises a therapeutic monoclonal antibody or a protein-based pharmaceutical.

Aspect 35. The method of aspect 32, wherein the target biomolecule comprises a target molecule for a small molecule drug.

Aspect 36. The method of aspect 32, wherein the target biomolecule comprises albumin.

Aspect 37. The method of aspect 1, wherein the method is conducted in a flow system, wherein the sample comprising the biological fluid is flowed through the flow system, and wherein the method is conducted sequentially on one or more subvolumes of fluid moving through the flow system.

Aspect 38. A system for evaluating a higher-order structure of a target biomolecule within a biological fluid, the system comprising:
(a) a fluidic channel for receiving an isolated biological fluid comprising a target biomolecule;
(b) a mixing portion for admixing one or more labeling reagents with the isolated biological fluid to produce a labeled target biomolecule, wherein the mixing portion is coupled to the fluidic channel for receiving the isolated biological fluid; and
(c) an automated device for receiving the labeled target biomolecule from the mixing portion.

Aspect 39. The system of aspect 38, further comprising an irradiation module for irradiating the one or more labeling reagents and the isolated biological fluid.

Aspect 40. The system of aspect 39, further comprising a quenching module for quenching the one or more labeling reagents, a degradation product thereof, or both.

Aspect 41. The system of aspect 38, further comprising a second mixing portion for admixing one or more buffers with the isolated biological fluid.

Aspect 42. The system of aspect 38, further comprising an in-line spectrophotometry module for analysis of biological fluid integrity, the spectrophotometry module being located between the fluidic channel for receiving the isolated biological fluid and the mixing portion.

Aspect 43. The system of aspect 42, wherein the in-line spectrophotometry module is configured to collect UV/vis spectra, fluorescence spectra, light scattering measurements, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Development of Whole Blood High Resolution Hydroxyl Radical Protein Footprinting (HR-HRPF) for Structural Pharmacology of Protein Pharmaceuticals

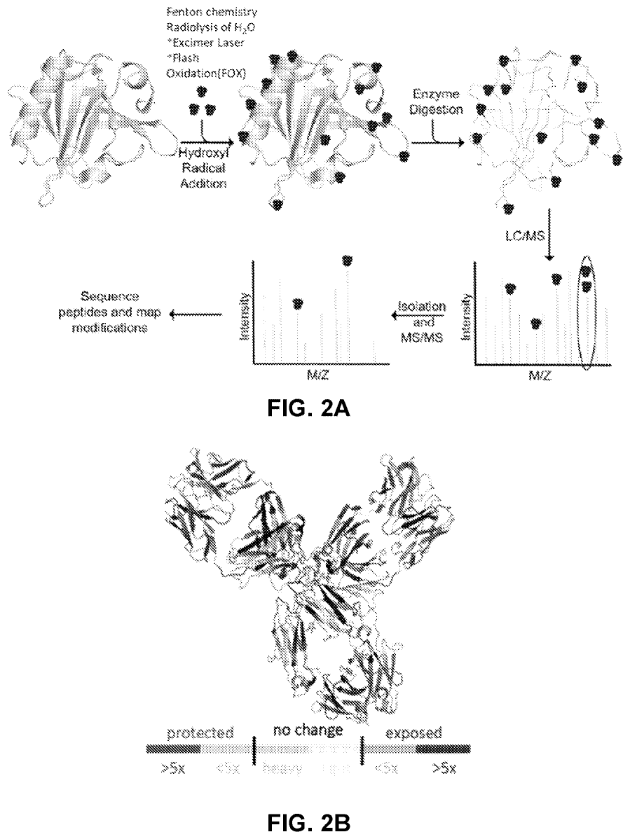

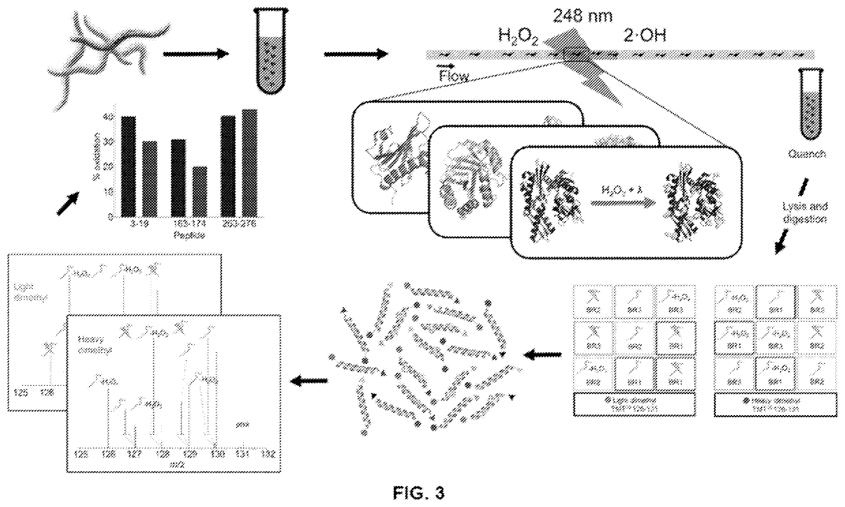

FIGS. 2A-2B show a schematic of a process for higher order structure characterization by hydroxyl radical protein footprinting (HRPF). As part of this process, structural mass spectrometry was used for characterization of protein structure in biologically relevant matrices. Fast photochemical oxidation of proteins (FPOP) of whole proteomes in living mammalian cells as well as in living C. elegans nematodes has been carried out. While mammalian whole organism FPOP is not currently feasible, a major issue in therapeutic drug discovery and pharmacology is the structure and interactions of drugs and drug targets in blood. Performing targeted HR-HRPF of a specific protein of interest also addresses one of the major issues of in cell and in vivo FPOP: complex peptide backgrounds and large dynamic ranges resulting in poor sequence coverage of most targets and difficulties in reproducibility. Herein, performance of these methods in stabilized whole blood is evaluated. A schematic of the FPOP process as disclosed herein is shown in FIG. 3.

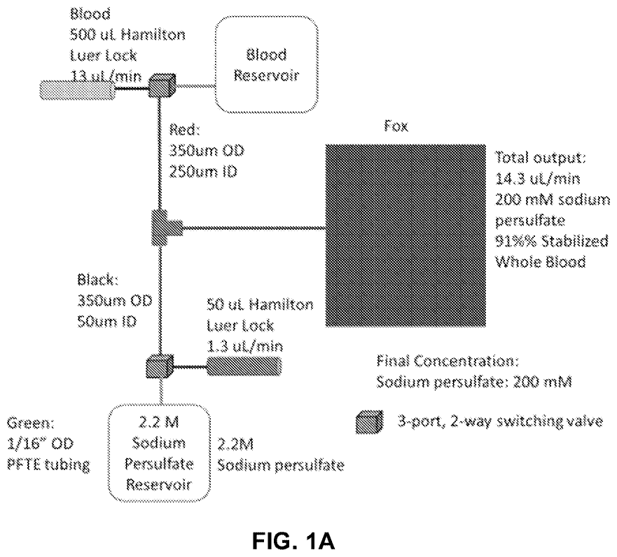

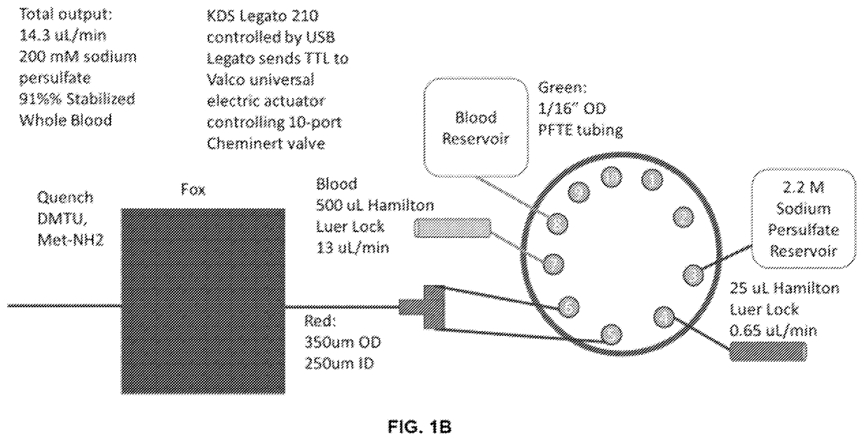

Development of flow system for whole blood FOX. Stabilized whole blood includes both cellular components (red blood cells, white blood cells, and platelets) as well as cell-free plasma components. While stabilization of the blood greatly slows coagulation, just the presence of the cellular components can cause cell clumping, which can lead to inconsistent protein labeling and system clogging. The initial design of a flow-based FOX system for labeling of whole blood is based upon an FPOP flow system initially designed for cell-based HRPF as shown in FIGS. 1A-1B, where a sheath flow is used to hydrodynamically focus cells to the center of the capillary lumen. Initially, a stock of bovine whole blood has been harvested for method development and stabilized with 1.5 g/L EDTA, which halts coagulation without causing hemolysis.

Protein oxidation is used to measure peroxide and small molecule diffusion at different regions of the flow. Ideally, hydrogen peroxide is applied in the sheath flow, delaying exposure of the blood to the hydrogen peroxide to prevent cell injury and peroxide conversion by catalase. Catalase activity can optionally be further delayed by the addition of an inhibitor such as 3-amino-1,2,4-triazole to the biological sample. Regular sample handling conditions including thawing, solvent exposure, and capillary pressure can lead to excess sample degradation before labeling can occur. A hemolysis assay may be used to measure if sample handling conditions are perturbing the native state of the blood. The hemolysis assay may be performed in-line prior to irradiation of the sample, or off-line using a control sample not exposed to radiation. Hydrogen peroxide concentration necessary for strong FOX oxidation can be determined using oxidation extent of both intracellular hemoglobin and spiked exogenous lysozyme as metrics of effective radical dose. Extracellular hemoglobin concentrations in non-anemic samples are <50 mg/L, while whole blood hemoglobin concentrations are between 120 and 180 g/L, showing that the vast majority of hemoglobin is intracellular and oxidation of hemoglobin should reflect the ability of peroxide to diffuse and penetrate the RBCs in the configuration used. Post-FOX centrifugation can also be used to separate cells from extracellular matrix, allowing further discrimination between labeling of intracellular and extracellular proteins. If necessary, chemical penetration enhancers can be used to increase penetration of peroxide into cells. To measure hemolysis under optimized conditions, release of hemoglobin from RBCs can be measured by spinning down cells and debris and measuring A415 of the ensuing supernatant, normalizing to complete hemolysis. In a typical experiment, hemolysis of red blood cells can be analyzed spectrophotometrically by measuring the hemoglobin released from lysed cells using a microplate reader.

The flow-based system allows for the optional discriminant labeling of extracellular proteins found in the plasma rather than labelling all proteins, intra- and extra-cellular, all under conditions with minimal hemolysis. This level of discrimination allows the selective study of protein structure extracellularly, separating out contributions from proteins that have been internalized into cells by any of a number of mechanisms. Discrimination between labeling of extracellular and intracellular proteins can be achieved through careful control of hydrogen peroxide diffusion time, hydrogen peroxide diffusion temperature, and the temperature of the samples. Under all conditions, cell integrity can be ensured to be maintained through measurement of hemolysis. This allows the development of FOX conditions for preferentially oxidizing extracellular proteins (as measured by exogenous protein spiked into whole blood) with an oxidation preference for extracellular proteins, as well as FOX conditions that allow oxidizing both intracellular and extracellular proteins as measured by oxidation of both exogenous spiked protein as well as hemoglobin. Ideally, experiments are performed under conditions that result in less than 20% hemolysis.

Performance of HR-HRPF of adalimumab paratope for TNFα. One of the primary envisioned applications of whole blood HR-HRPF is the structural interrogation of biotherapeutics in mammalian whole blood, in order to understand conformational changes and protein interactions in biologically and medically relevant contexts and detect interactions and conformational changes unanticipated based on in vitro experiments. Using in vitro target protein HRPF results, including, but not limited to, those for adalimumab, targeted MS/MS methods can be designed using observed retention time and m/z for both unoxidized and oxidized versions of the target proteins and/or peptides. Recombinant target proteins can be spiked into whole at concentrations starting at 165 nM (5× the peak therapeutic concentration of adalimumab from the current recommended maintenance human dosing) and going down to 264 pM, wherein the amounts can be modified for different target proteins. The limit of detection of unoxidized target protein spiked in whole blood can be tested, focusing on maximizing sequence coverage. This system can then be applied to bovine whole blood that has target protein spiked in at concentrations at which high sequence coverage can be generated. Tandem mass tag (TMT) can be used to improve reproducibility and reliability of sequence coverage, multiplexing all replicates as well as the no peroxide and no flash negative controls. LC-MS/MS conditions as well as FOX conditions can be optimized to maximize oxidation and sequence coverage under conditions that do not cause high levels of hemolysis. The limit of quantification can be determined wherein results generated are comparable to in vitro published results.

In order to map the paratope of adalimumab, the system described above can be applied to rodent whole blood that has been extracted from C57BL/6 mice. Non-terminal withdrawal collects blood in 200 μL volumes. A TNFα Simplex kit (Invitrogen) can be used with the Luminex FlexMap3D to quantify TNFα from these cell lines. As normal TNFα levels are considerably lower than peak adalimumab dosing (TNFα concentration in health humans is generally about 4 pM), exogenous TNFα was spiked in to concentrations equimolar to adalimumab paratopes in one set of samples, and the exogenous TNFα was left out in the other. Whole blood FOX was used to oxidize both sets of samples, using TMT-10 to multiplex quadruplex sets from both conditions as well as two negative controls. By this method, protection of the adalimumab paratope should be identified by the differential footprint between TNFα-spiked and naturally occurring levels of TNFα. Similarly, footprinting of adalimumab in whole blood can be compared with that measured in vitro to determine if other regions of adalimumab show protection upon presence in whole blood, indicating topographical differences induced by the presence in whole blood.

Performance of comparative HR-HRPF of adalimumab subclasses in bovine blood. One of the goals of whole blood FOX is to develop a tool that can differentiate between conformations and interactions in the biologically complex system of whole mammalian blood. Herein, the technology is validated using a system of exogenously added adalimumab to determine figures of merit for the method. The vast majority of biotherapeutics approved or in development are IgG1 molecules; however, there are four major subclasses of IgG. These subtypes differ in their constant region, most notably in the hinge and upper CH2 domains. Due to these structural differences, they engage different Fcγ receptors and have different levels of C1q binding, among other differences in function and interactions with potential implications in biomedically important phenomena such as antibody-dependent enhancement of pathogenesis. A series of adalimumab-derived IgG subtypes was developed for IgG1-4. 164.5 μg of these subtypes were administered into C57BL/6 mice subcutaneously, an equivalent to the human dose of 0.67 mg/kg. Blood was non-terminally drawn (200 μL) six hours post injection (estimated to be near Tₘₐₓ). Whole blood FOX HRPF was performed using instrumentation and methods as described above. In vitro HRPF of each subclass can be used to develop a targeted LC-MS/MS method, and TMT multiplexing can be used to improve throughput, reproducibility, and reliable sequence coverage. All subclasses can be compared to IgG1 as a baseline to determine differences in topography in whole blood, and these differences can be compared with those measured in vitro. Footprints of each subclass in whole blood can be generated that are comparable to those generated in vitro using existing well-characterized methods for FOX HRPF, while observing any blood-induced differences in protein topography due to conformation and/or interactions.

If the FOX system proves unsuitable, whole blood HRPF can be moved to high energy KrF excimer laser-based FPOP platforms. In the event that catalase activity proves too rapid for hydrogen peroxide-based FOX oxidation, alternative mixed hydroperoxides can be used as photoactivatable hydroxyl radical precursors that are poor substrates for catalase. In the event that TNFα levels in C57BL/6 mice are found to be too high to allow for footprinting of the unbound adalimumab in whole blood based on Luminex quantification, a TNFα-free blood can be produced using a TNFα strain generated from the C57BL/6 background (RRID: IMSR_JAX:00540). A Protein G column post-oxidation can be used, if necessary, to concentrate for all subclasses of the present IgG biotherapeutics, enabling generation of a sufficient signal for HRPF.

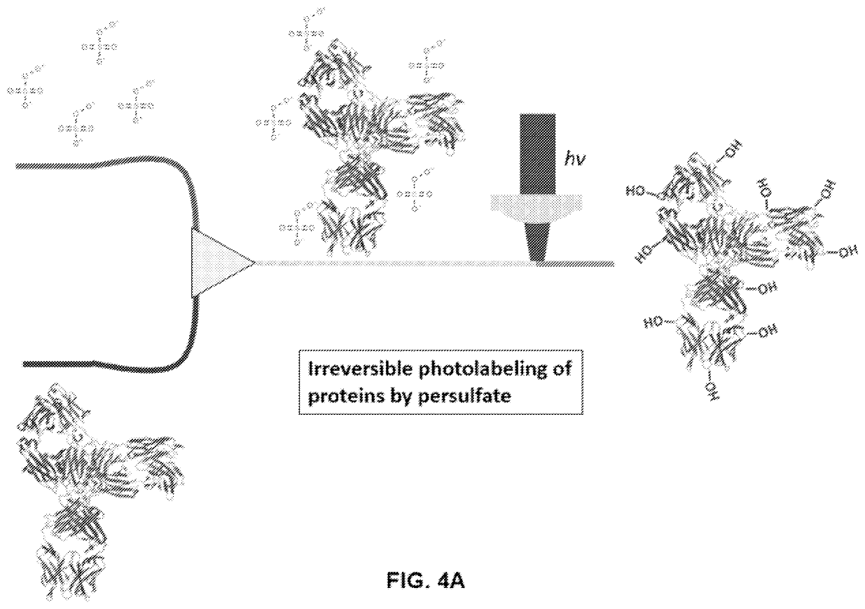

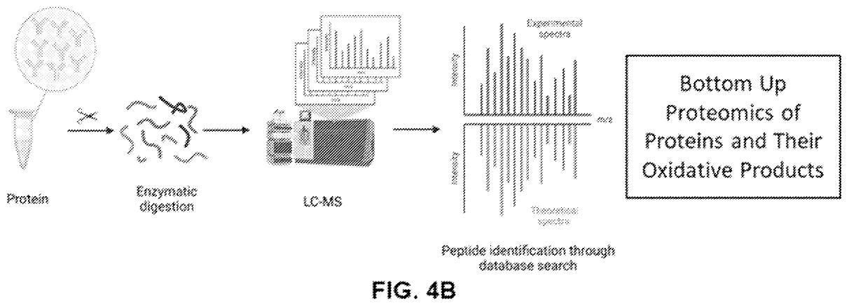

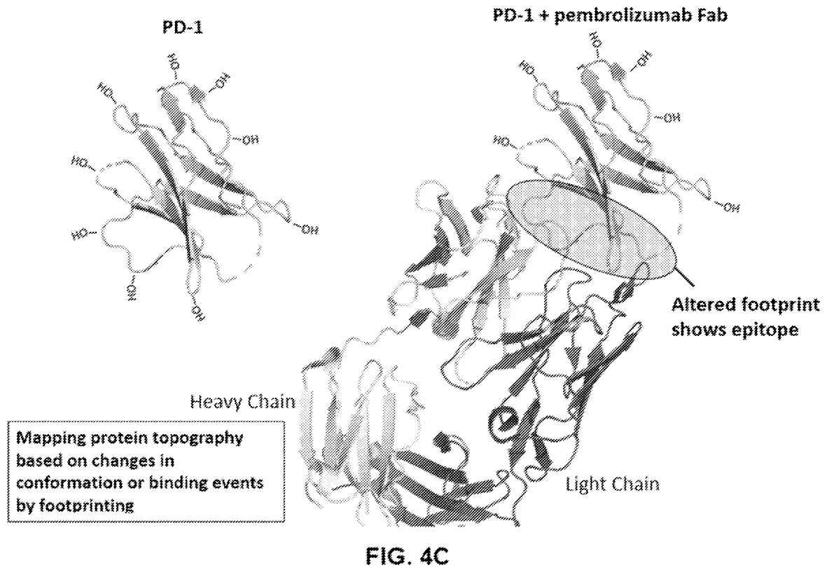

Vertebrate animals. All experiments involving the use of animals are evaluated by the Institutional Animal Care and Use Committee (IACUC) of the University of Mississippi to ensure compliance with IACUC, NIH, and state regulations governing the use of animals in laboratory research settings. Male C57Bl/6 mice were used. Mice were group housed at an accredited and IACUC-monitored facility at the University of Mississippi. Mice entered the study at 8 weeks of age, to increase the time at which the mice remain healthy and available for nonterminal blood draws. Untreated mice had non-terminal blood draws of 200 μL volumes per mouse immediately stabilized with 1.5 g/L EDTA, to be used as an ex vivo matrix for analysis of spiked material. FIGS. 4A-4C show a schematic of an ex vivo FOX-HRPF process for analyzing proteins in whole blood.

In some experiments, mice were dosed with 165 μg of adalimumab of one of up to four subclasses, each by subcutaneous injection. Six hours post-injection, each mouse had a nonterminal 200 μL blood draw, immediately stabilized by 1.5 g/L EDTA.

Rodents were monitored by qualified personnel for signs of pain or distress during the study. Personnel are trained to use only sharp needles and non-invasive handling techniques. Following the completion of the study, rodents were euthanized with exposure to carbon dioxide or an overdose of sodium pentobarbital.

### Example 2: Experimental Results

### Materials

Equine myoglobin, N,N′-Dimethylthiourea (DMTU), hydroxylamine, sucrose, and sodium persulfate were obtained from Sigma Aldrich Corp. (St. Louis, MO). Triton X-100, imidazole, and EDTA were purchased from VWR International (Radner, PA). Adenine, 3-amino-1:2:4-triazole (3AT), aminoguanidine, hydrogen peroxide (30%), MS-grade trypsin, and thiosemicarbazide (TSC) were purchased from Thermo Fisher Scientific (Waltham, MA). Methionine amide was purchased from Bachem (Bubendorf, Switzerland). Ferrules, capillary sleeves, Luer lock to %-28 adapters, and microtees were purchased from IDEX Health & Science (Oak Harbor, WA). Syringes and 3-port manual valves were purchased from Hamilton (Reno, NV). Bovine blood was generously provided by Home Place Pastures (Como, MS). Murine blood was obtained from the University of California, San Diego Animal Facilities.

### Foam Assay

Four previously reported catalase inhibitors (hydroxylamine, 3AT, aminoguanidine, and TSC) were assessed in blood via a test tube foam assay modified from Iwase et al, which correlates trapped catalase-generated oxygen gas to catalase activity. Blood was collected during routine exsanguination for beef production, immediately stabilized with EDTA (1.8 mg/mL), and flash frozen with dry ice/ethanol. Thawed blood was incubated with an inhibitor in a test tube, into which 50 μL of 2% Triton X-100 and 100 μL of 30% H₂O₂ were spiked. Foam height was recorded. To optimize inhibition, inhibitor concentration, incubation duration and temperature, and volumetric (v/v) ratio of blood to inhibitor stock were manipulated.

### In Vitro Oxidation and Dosimetry

Samples with 1 mM adenine and 50 mM Na₂S₂O₈ were prepared in triplicate and irradiated at 2 Hz by a FOX photolysis system (GenNext Technologies, Inc., Half Moon Bay, CA). Real-time changes in adenine absorbance were monitored at 265 nm (Abs₂₆₅ₙₘ) by the inline UV spectrophotometer of the FOX system. UV lamp voltage was increased in 100 V increments from 600 to 900 V. Other triplicate samples were irradiated at 900 V and contained varied sodium persulfate concentrations (0, 50, 75, 100, 125, 150, 175 mM) or varied sucrose concentrations (0, 25, 50, 75, 100, 125 mM). A decrease in Abs₂₆₅ₙₘ following irradiation indicates radical scavenging by adenine.

The efficiency of the sodium persulfate quench was evaluated by injecting the above samples at 0 V, and by injecting samples of 100 mM persulfate, 1 mM adenine, and 25 mM sodium phosphate (pH 7.2) into the FOX, irradiating at 900 V, and collecting 20 μL of sample in a 20 μL quench solution of 5 μM myoglobin, 5 μM GluB, 200 mM imidazole, 200 mM DMTU, 70 mM Met-NH₂, and 25 mM sodium phosphate (pH 7.2). Protein labeling samples contained 5 μM myoglobin, 5 μM GluB, 1 mM adenine, 25 mM sodium phosphate (pH 7.2), and 100 mM sodium persulfate. The samples underwent FOX irradiation at 900 V and 20 μL of sample were collected in a 20 μL quench solution of 200 mM imidazole, 200 mM DMTU, 70 mM Met-NH₂, and 25 mM sodium phosphate (pH 7.2).

A final concentration of 100 mM Tris, pH 8.0 and 1 mM CaCl₂ was added to the samples and heated at 95° C. for 15 min and immediately placed on ice for 2 min. 333 ng trypsin was added to the samples and incubated at 37° C. with rotation for 14 hours. A final concentration of 0.1% formic acid was added to the samples to stop the trypsin activity. The samples were injected via autosampler on a Dionex Ultimate 3000 nano-LC system coupled to an Orbitrap Fusion Tribrid mass spectrometer (Thermo Scientific, San Jose, CA). Modification products were manually identified using Thermo Xcalibur Qual Browser. The number of oxidation and other modification events per peptide was calculated using Equation 1:

$$Extent of Oxidation = \frac{\text{?} ( ( XIC Peak Area for {OxLevel}_{n} ) ( n ) )}{\begin{matrix}( XIC Peak Area Unmodified Peptide ) + \\ \text{?} ( XIC Peak Area for {OxLevel}_{n} )\end{matrix}} \qquad ( Eq . 1 )$$

$$\text{?} \text{indicates text missing or illegible when filed}$$

The percent of unmodified peptides was calculated with Equation 2:

$$( Eq . 2 )$$

$$Percent unmodified = \frac{XIC Peak Area of Unmodi \text{?} fied peptide}{\begin{matrix}( XIC Peak Area of Unmod \text{?} fied peptide ) + \\ \sum ( XIC Peak Area for OxLevel n )\end{matrix}}$$

$$\text{?} \text{indicates text missing or illegible when filed}$$

### Flow System Design

A schematic of an exemplary flow system for inline mixing of sodium persulfate with stabilized whole mammalian blood is shown in FIG. 1. A 500 μL syringe and a 50 μL syringe were loaded side-by-side on a Legato 210 syringe pump (KD Scientific, Holliston MA). Each syringe pump connected directly to a 3-port manual switching valve through a Luer lock to ¼-28 adapter. One port of each switching valve had a short 1/16″ OD PFTE tubing going into a reservoir containing either stabilized whole blood (connected to the 500 μL syringe) or 2.2 M sodium persulfate (connected to the 50 μL syringe). The third port of each valve was connected to a 29 cm long fused silica capillary (360 μM OD, 250 μm ID for blood syringe; 360 μM OD, 50 μm ID for persulfate syringe) which went to opposite ports of a PEEK MicroTee. The third port of the microtee was connected to a 68 cm length of UV transparent fused silica capillary (360 μM OD, 250 μm ID) which was passed through the flash module, dosimetry module and sample collection module of a Fox Photolysis System (GenNext Technologies, Inc, Half Moon Bay, CA). The system was manually primed through both Luer lock adapters to remove air from the lines and valve prior to operation.

### Mixing Imaging

Liquid blue and red food coloring (local grocery store) was mixed at various concentrations and measured to ensure proper levels of absorbance. Final concentrations of 1 mL red dye in 10 mL of water and 100 μL of blue dye in 20 mL of water gave appropriate absorbance intensities to allow imaging of mixing when blue dye is used in the 500 μL syringe and red dye is used in the 50 μL syringe. The outflow from the microtee was imaged at various distances from the tee in brightfield mode after white balancing according to manufacturer's instructions, using the 4× objective lens. Images were analyzed using the ColourDeconvolution2 plugin for ImageJ. Images were obtained for the capillary filled with only red dye and with only blue dye, and these images were used to select the colors for deconvolution. Histograms for each color versus distance were then plotted for each image to determine when mixing was complete.

### In Blood Protein Oxidation

The syringe pump was programmed to withdraw a set amount of sample and persulfate from the reservoirs, and then deliver 13.6 μL/min of blood and 1.36 μL/min of sodium persulfate through the Fox Photolysis System. All modules of the Fox system were operated in a manual mode at a lamp repetition rate of 2 Hz for two minutes per sample, with the delay time between flash start and sample collection start set to two minutes based on visual analysis of the time required for blood to reach each module through the transparent capillary. All samples were collected in a tube containing 200 mM DMTU, 200 mM imidazole, and 70 mM methionine as a persulfate quench in a volume equal to that of the collected sample (30 μL).

### Selection of Stable Radical Precursor for Whole Blood

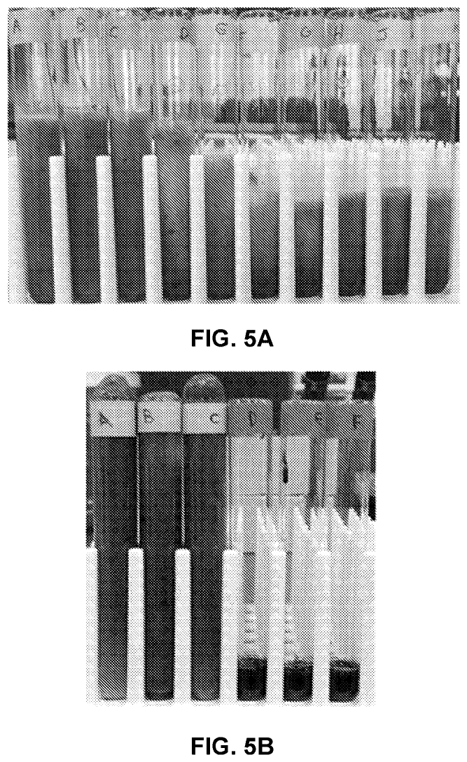

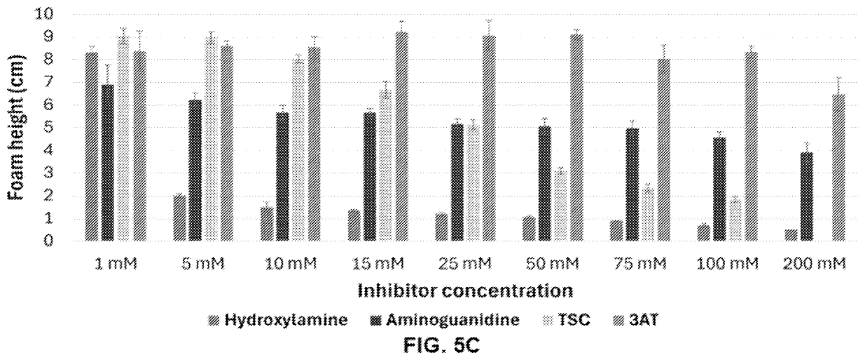

Most previous cell-based and in vivo radical footprinting experiments have used hydrogen peroxide-based FPOP for fast radical generation. However, blood notably displays high catalase activity, which prevents use of hydrogen peroxide as a radical precursor. Four catalase inhibitors were tested to determine if peroxidase activity in whole blood could be sufficiently reduced without altering the gross physiological properties of the blood sample. Gas generation in blood from sodium persulfate, previously utilized for in-cell FPOP, was also assessed. Of the four inhibitors, HA was significantly more effective, with a 95% reduction in catalase activity at a final 200 mM concentration, 5:1 v/v blood to inhibitor stock, and room temperature as seen in FIGS. 5A-5C. Longer incubation and reduced temperature improved inhibition (data not shown). At a more physiologically relevant 20:1 v/v, inhibition was significantly reduced. Moreover, TSC and HA induced gross changes in blood coloration and constitution, clearly disrupting the native blood environment. Ultimately, none of the four inhibitors prevented foam generation blood at concentrations compatible with physiological conditions. Barring the identification of some other effective inhibitor, HRPF is likely not feasible in whole blood with H₂O₂ as a radical precursor. Conversely, when sodium persulfate was spiked into blood at final 200 mM concentration (FIGS. 5A-5C), no gas generation or gross changes were observed.

Evaluation of Sodium Persulfate Radical Footprinting using the FOX System

Adenine dosimetry. Adenine was investigated as a SO₄—· dosimeter since it had been demonstrated effective for hydroxyl radical dosimetry. Adenine dosimetry records the loss of UV absorbance at 265 nm that occurs when adenine is oxidized, allowing adenine UV absorbance to measure changes in both radical generation and scavenging. As sulfate and hydroxyl radicals are broadly reactive and can be consumed by solutes like buffers and excipients, real-time dosimetry allows for compensation to ensure that analytes receive equivalent radical doses. Accordingly, the linearity of the in-line UV absorbance of 1 mM adenine was examined under various conditions.

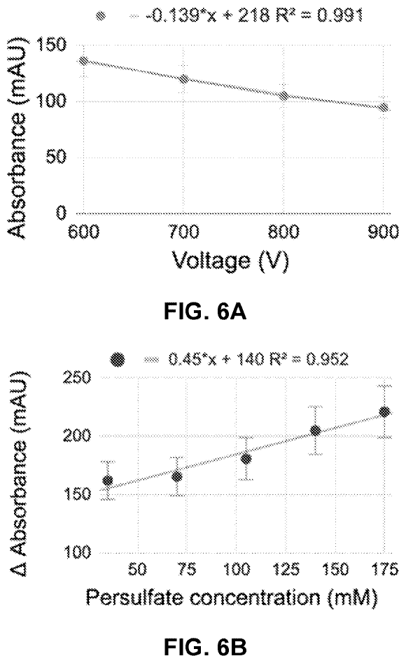

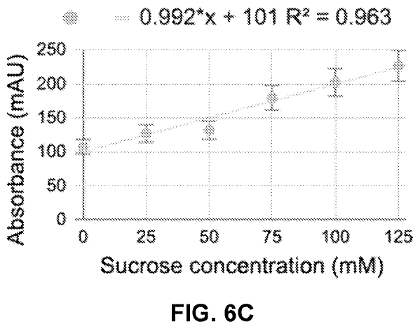

Adenine dosimeter response was assessed first in response to an increase in the effective radical dose, achieved via increased light fluence (as modulated by flash voltage) or increased radical precursor concentration. Abs₂₆₅ₙₘ decreased by 0.139 mAU/V as lamp voltage was increased from 600 V to 900 V in the presence of 50 mM persulfate (FIG. 6A). Adenine dosimetry obtained with varied radical precursor concentration, however, is confounded by the intrinsic Abs265 nm of sodium persulfate. Thus, Abs265 nm was recorded for triplicate samples containing 1 mM adenine and varied persulfate concentration that were irradiated at 0 V or 900 V. These values were compared to find the magnitude of the decrease in Abs₂₆₅ₙₘ, (hereafter denoted by ΔAbs₂₆₅ₙₘ) controlling for absorbance due to increased persulfate concentration. As seen in FIG. 6B, ΔAbs₂₆₅ₙₘ increased by 0.45 mAU per mM persulfate. In order to determine the reliability of adenine dosimetry in the presence of a scavenger that competes for radicals, sucrose concentration was increased across triplicate samples with a lamp voltage of 900 V and 50 mM sodium persulfate. This yielded a linear response within the range tested, with Abs₂₆₅ₙₘ increasing by 0.992 mAU per mM sucrose (FIG. 6C).

These dosimetry results show that adenine can be a highly reliable SO₄—· dosimeter in the appropriate buffer systems, allowing for the real-time compensation of in vitro conditions across sulfate RPF experiments. However, compound changes involving both a change in persulfate concentration with a change in either lamp voltage or scavenger concentration need to be careful to account for the inherent Abs₂₆₅ₙₘ of persulfate. Lamp voltage should only be changed for compensation efforts in persulfate-based labeling.

Quench. Recent observations have indicated that sulfate RPF is not effectively quenched by standard HRPF quenching protocols as previously published, with background modification still observed when standard quench protocols were used. An effective quench is critical as high numbers of modifications can cause protein unfolding, allowing secondary radicals to cause extraneous modifications and compromise validity. Imidazole-based compounds have previously been demonstrated as potent scavengers for SO₄—· and resulting secondary oxidants. 200 mM imidazole was incorporated into a standard quench containing 200 mM DMTU and 70 mM Met-NH₂, which was demonstrated to be effective with photoactivated H₂O₂. Quench efficacy was assessed at flash lamp voltages of 900 V and 0 V. In order to isolate modifications that occur in the quench and during subsequent sample preparation, myoglobin and model peptide GluB was spiked into the quench buffer and the persulfate solution was illuminated in the Fox system at a flash lamp voltage of 900 V to initiate secondary oxidant creation. In the 0 V condition, myoglobin was injected into the FOX with persulfate before collection in a quench. This allows comparison of modification rates to discern whether pre-FOX sample preparation and in-line flow is a significant source of oxidation. To determine the efficacy of imidazole as a scavenger, the above was also conducted with quenches only containing DMTU and Met-NH₂.

The results from these experiments are shown in Table 1. GluB and seven peptides from myoglobin were detected. During electrospray ionization, significant in-source oxidation occurred for the peptide 2-17, even in samples lacking Na₂S₂O₈; this in-source oxidation can be differentiated from pre-existing oxidation by the perfect LC co-elution of the in-source oxidation product with the unmodified peptide. For quenches containing 200 mM imidazole, there was no significant difference in peptide modification with or without sodium persulfate. Moreover, there was not a significant increase in oxidation when myoglobin was injected in-line with Na₂S₂O₈ as reported previously. These results indicate that a quench containing DMTU, methionine and imidazole is sufficient to prevent background oxidation in persulfate-based RPF experiments in the Fox system. Some peptides in quenches that only contained DMTU and methionine did display a significant increase in background oxidation, replicating previous unpublished observations. For instance, the peptide 120-134 was modified three to four-fold more in quenches lacking imidazole. This suggests that the imidazole-containing quench may be most critical for peptides that are more readily oxidized.

TABLE 1
Percent of Peptides Modified in Quench Across Different Quench Conditions

| | Imidazole Quench Set | | | Non-Imidazole Quench Set | | |
|---|---|---|---|---|---|---|
| Peptide | | | 900 V, 100 | | | 900 V, 100 |
| name or | | 900 V, 100 | mM | | 900 V, 100 | mM |
| position | 900 V, 0 mM | mM | persulfate, | 900 V, 0 mM | mM | persulfate, |
| within MB | persulfate | persulfate | no quench | persulfate | persulfate | no quench |
| 2-17¹ | 9.88 ± 2.03 | 9.99 ± 0.48 | 20.25 ± 4.71 | 9.53 ± 2.46 | 13.77 ± 0.62 | 42.55 ± 7.26 |
| 18-32 | 0.06 ± 0.02 | 0.27 ± 0.24 | 32.91 ± 5.71 | 0.07 ± 0.01 | 0.01 ± 0.01 | 38.24 ± 5.81 |
| 33-43 | 0.05 ± 0.03 | 0.03 ± 0.03 | 34.75 ± 7.93 | 0.02 ± 0.01 | 0.01 ± 0.00 | 30.00 ± 5.73 |
| 58-64 | 0.02 ± 0.02 | 0.07 ± 0.04 | 2.47 ± 0.30 | 0.20 ± 0.01 | 0.02 ± 0.00 | 3.28 ± 0.38 |
| 65-78 | 0.31 ± 0.05 | 0.26 ± 0.02 | 20.33 ± 2.91 | 0.24 ± 0.01 | 0.20 ± 0.01 | 25.71 ± 4.89 |
| 120-134 | 0.86 ± 0.03 | 0.82 ± 0.19 | 93.03 ± 4.68 | 0.86 ± 0.05 | 3.37 ± 0.09 | 95.87 ± 0.72 |
| 135-140 | 0.21 ± 0.03 | 0.17 ± 0.04 | 5.64 ± 1.38 | 0.18 ± 0.02 | 0.21 ± 0.05 | 5.38 ± 2.12 |
| GluB | 0.32 ± 0.05 | 0.29 ± 0.01 | 28.28 ± 2.74 | 0.33 ± 0.05 | 0.32 ± 0.03 | 28.98 ± 3.08 |
| Peptide | Imidazole Quench Set | | | Non-Imidazole Quench Set | | |
| name or | | | 0 V, 100 mM | | | 0 V, 100 mM |
| position | 0 V, 0 mM | 0 V, 100 mM | persulfate, | 0 V, 0 mM | 0 V, 100 mM | persulfate, |
| within MB | persulfate | persulfate | no quench | persulfate | persulfate | no quench |
| 2-17¹ | 10.59 ± 2.21 | 11.50 ± 0.52 | 15.89 ± 2.94 | 8.47 ± 0.02 | 14.91 ± 1.47 | 10.56 ± 1.92 |
| 18-32 | 0.02 ± 0.02 | 0.03 ± 0.04 | 56.94 ± 3.57 | 0.05 ± 0.01 | 0.06 ± 0.02 | 67.04 ± 3.45 |
| 33-43 | 0.07 ± 0.03 | 0.00 ± 0.00 | 47.61 ± 4.37 | 0.04 ± 0.01 | 0.12 ± 0.05 | 52.83 ± 1.48 |
| 58-64 | 0.02 ± 0.01 | 0.01 ± 0.01 | 1.52 ± 0.88 | 0.01 ± 0.00 | 0.05 ± 0.23 | 2.57 ± 0.16 |
| 65-78 | 0.28 ± 0.18 | 0.33 ± 0.03 | 20.27 ± 5.54 | 0.34 ± 0.01 | 0.50 ± 0.04 | 32.38 ± 2.00 |
| 120-134 | 1.25 ± 0.07 | 0.90 ± 0.03 | 76.83 ± 10.83 | 1.51 ± 0.08 | 4.46 ± 0.24 | 79.91 ± 4.80 |
| 135-140 | 0.15 ± 0.07 | 0.11 ± 0.01 | 7.41 ± 1.71 | 0.15 ± 0.02 | 0.19 ± 0.02 | 7.86 ± 0.61 |
| GluB | 0.27 ± 0.05 | 0.47 ± 0.09 | 29.31 ± 5.75 | 0.47 ± 0.27 | 0.29 ± 0.03 | 27.13 ± 0.43 |

Error reported as ± 1 SD.
¹Significant in-source oxidation was detected.

### In Vitro Protein Oxidation

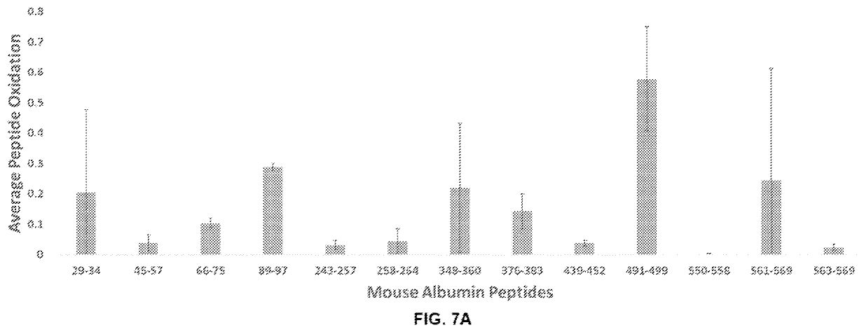

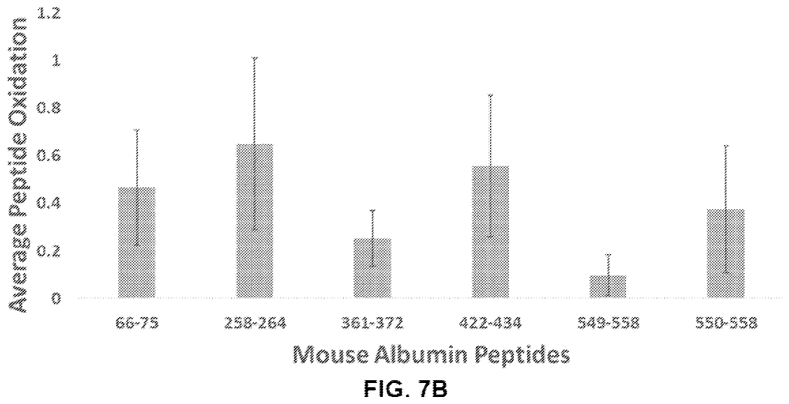

Results for in vitro FOX footprinting of mouse albumin are seen in FIGS. 7A-7B.

### Sulfate Radical Footprintinq in Whole Blood

Creation and characterization of semi-automated sample handling and mixing system. In order to simplify handling of stabilized whole blood, minimize sample incubation time with the persulfate radical precursor and improve reproducibility of results, a semi-automated liquid handling system as shown in FIGS. 1A-1B and described in the Experimental Section was generated. Briefly, blood and sodium persulfate are drawn into two separate syringes operated by the same programmable syringe pump. A 10:1 difference in volume per linear unit allows for 10:1 sample dispensing by using a 500 μL syringe for stabilized whole blood and a 50 μL syringe for persulfate. Manual three-port valves are used to switch samples from withdraw from reservoirs to infusion into the flow system. Blood and persulfate are introduced into a PEEK Microtee at opposite ports and allowed to mix within the tee and in the outflow capillary at a rate of 13.6 μL/min of blood flow and 1.36 μL/min of persulfate flow, for a total flow rate of 14.96 μL/min.

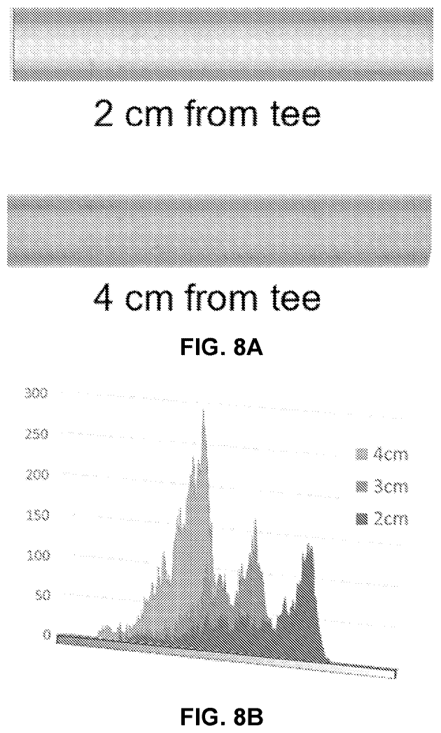

In order to ensure complete mixing of blood and persulfate prior to illumination by the Fox Photolysis System, the mixing of blue and red dye mixed at 10:1 ratio was measured as a function of distance from the mixing tee. Sample images are shown in FIG. 8A. Segregation of the red dye color (representing the persulfate) can be observed clearly at 2 cm from the tee, with the color evening at 4 cm from the tee. This segregation was quantified using the histogram function to measure color intensity across the capillary (FIG. 8B). Color was found to reach homogeneity at 4 cm from the tee, consistent with manual visual analysis of the images. The flow system was set up to place illumination region of Fox Flash Unit at a distance of 6 cm from the mixing tee to ensure homogenous mixing of the persulfate and blood.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abbvie Inc. (Revised 2021) Humira (adalimumab) [package insert]. U.S. Food and Drug Administration website.
2. Abolhasani Khaje N et al (2020) Rapid Quantification of Peptide Oxidation Isomers From Complex Mixtures. Anal Chem 92(5):3834-3843.
3. Abuin E, et al (2012) Diffusion of hydrogen peroxide across DPPC large unilamellar liposomes. Chem Phys Lipids 165(6):656-661.
4. Aebi, Hugo. “Catalase.” Methods of enzymatic analysis. Academic press, 1974. 673-684.
5. Aprahamian M L et al (2019) Utility of Covalent Labeling Mass Spectrometry Data in Protein Structure Prediction with Rosetta. J Chem Theory Comput 15(5):3410-3424. PMCID:PMC6520167
6. Aprahamian M L, et al (2018) Rosetta Protein Structure Prediction from Hydroxyl Radical Protein Footprinting Mass Spectrometry Data. Anal Chem 90(12):7721-7729. PMCID:PMC6008241
7. Bezerra F F, et al (2019) Conformational properties of 1-fucose and the tetrasaccharide building block of the sulfated 1-fucan from Lytechinus variegatus. J Struct Biol:107407.
8. Biehn S E et al (2021) Accurate protein structure prediction with hydroxyl radical protein footprinting data. Nat Commun 12(1):341. PMCID:PMC7804018
9. Bournazos S, et al (2020) The role of IgG Fc receptors in antibody-dependent enhancement. Nat Rev Immunol 20(10):633-643. PMCID:PMC7418887
10. Buxton G V, et al (1988) Critical-Review of Rate Constants for Reactions of Hydrated Electrons, Hydrogen-Atoms and Hydroxyl Radicals (·OH/·O—) in Aqueous-Solution. J Phys Chem Ref Data 17(2):513-886.
11. Chauhan U P. Glutathione as an inhibitor of trypsin induced proteolysis. Indian J Exp Biol. 1989 May; 27(5):472-3.
12. Chea E E et al (2018) Modifications generated by fast photochemical oxidation of proteins reflect the native conformations of proteins. Protein Sci 27(6):1047-1056. PMCID:PMC5980583
13. Cheetham J C, et al (1992) Refinement of an enzyme complex with inhibitor bound at partial occupancy. Hen egg-white lysozyme and tri-N-acetylchitotriose at 1.75 A resolution. J Mol Biol 224(3):613-628.
14. Cheng M, et al (2017) Laser-Initiated Radical Trifluoromethylation of Peptides and Proteins: Application to Mass-Spectrometry-Based Protein Footprinting. Angewandte Chemie (International ed 56(45):14007-14010. PMCID:PMC5663992
15. Cheng M, et al (2020) Fast Protein Footprinting by X-ray Mediated Radical Trifluoromethylation. J Am Soc Mass Spectrom 31(5):1019-1024. PMCID:PMC7486011
16. Cheng Z, et al (2021) Self-Organized Amphiphiles Are Poor Hydroxyl Radical Scavengers in Fast Photochemical Oxidation of Proteins Experiments. J Am Soc Mass Spectrom 32(5):1155-1161.
17. Cheng Z, et al (2022) Structural Analysis of Phosphorylation Proteoforms in a Dynamic Heterogeneous System Using Flash Oxidation Coupled In-Line with Ion Exchange Chromatography. DOI: 10.1101/2022.10.04.510855. Version 1, cited 10/12/2022. (preprint).
18. Damas P, et al (1989) Tumor necrosis factor and interleukin-1 serum levels during severe sepsis in humans. Crit Care Med 17(10):975-978.
19. de Taeye S W, et al (2019) The Ligands for Human IgG and Their Effector Functions. Antibodies (Basel) 8(2). PMCID:PMC6640714
20. Drake Z C, et al (2022) Protein Complex Prediction Using Rosetta, AlphaFold, and Mass Spectrometry Covalent Labeling. (preprint).
21. Espino J A et al (2019) Illuminating Biological Interactions with in Vivo Protein Footprinting. Anal Chem 91(10):6577-6584. PMCID:PMC6533598
22. Espino J A et al (2020) In Vivo Hydroxyl Radical Protein Footprinting for the Study of Protein Interactions in Caenorhabditis elegans. J Vis Exp (158). PMCID:PMC7897402
23. Espino J A, et al (2015) In Cell Footprinting Coupled with Mass Spectrometry for the Structural Analysis of Proteins in Live Cells. Anal Chem 87(15):7971-7978.
24. Espino J A, et al (2020) Chemical Penetration Enhancers Increase Hydrogen Peroxide Uptake in C. elegans for In Vivo Fast Photochemical Oxidation of Proteins. J Proteome Res 19(9):3708-3715. PMCID:PMC7861136
25. Espino J A, et al (2020) In Vivo Fast Photochemical Oxidation of Proteins Using Enhanced Multiplexing Proteomics. Anal Chem 92(11):7596-7603. PMCID:PMC7815197
26. Fry S C (1998) Oxidative scission of plant cell wall polysaccharides by ascorbate-induced hydroxyl radicals. Biochem J 332 (Pt 2):507-515. PMCID:1219507
27. Garrison W M (1987) Reaction Mechanisms in the Radiolysis of Peptides, Polypeptides, and Proteins. Chem Rev 87(2):381-398.
28. Gau B C et al (2009) Fast photochemical oxidation of protein footprints faster than protein unfolding. Anal Chem 81(16):6563-6571. PMCID:PMC3164994.
29. Gau B C, et al. Sulfate radical anion as a new reagent for fast photochemical oxidation of proteins. Anal Chem. 2010 Sep. 15; 82(18):7821-7. doi: 10.1021/ac101760y.
30. Hambly D M et al (2005) Laser flash photolysis of hydrogen peroxide to oxidize protein solvent-accessible residues on the microsecond timescale. J Am Soc Mass Spectrom 16(12):2057-2063.
31. Hamdan S, et al (2022) Cytomegalovirus immune evasion sets the functional avidity threshold for protection by CD8 T cells. Med Microbiol Immunol.
32. Han V, et al (2010) A comparative study of common techniques used to measure haemolysis in stored red cell concentrates. Vox Sang 98(2):116-123.
33. Hu S, et al (2013) Comparison of the inhibition mechanisms of adalimumab and infliximab in treating tumor necrosis factor alpha-associated diseases from a molecular view. J Biol Chem 288(38):27059-27067. PMCID:PMC3779706
34. Huang R, et al (2011) LC-MS(n) analysis of isomeric chondroitin sulfate oligosaccharides using a chemical derivatization strategy. J Am Soc Mass Spectrom 22(9):1577-1587. PMCID:3187560
35. Huang R, et al (2013) An approach for separation and complete structural sequencing of heparin/heparan sulfate-like oligosaccharides. Anal Chem 85(12):5787-5795. PMCID:3725598
36. Huang R, et al (2016) De Novo Sequencing of Complex Mixtures of Heparan Sulfate Oligosaccharides. Anal Chem 88(10):5299-5307. PMCID:PMC5068567
37. Huang R R et al (2010) Multi-Step Derivatization for the Structural Analysis of Glycosaminoglycan Oligomers by Reverse Phase LC-Ms. Glycobiology 20(11):1504-1504.
38. Huang W, et al (2015) Quantitative mapping of protein structure by hydroxyl radical footprinting-mediated structural mass spectrometry: a protection factor analysis. Biophys J 108(1):107-115. PMCID:4286602
39. Itakura Y, et al (2007) Systematic comparison of oligosaccharide specificity of Ricinus communis agglutinin I and Erythrina lectins: a search by frontal affinity chromatography. J Biochem 142(4):459-469.
40. Iwase T, et al. A simple assay for measuring catalase activity: a visual approach. Sci Rep. 2013 Oct. 30; 3:3081. doi: 10.1038/srep03081.
41. Johnson D J et al (2003) Crystal structure of antithrombin in a heparin-bound intermediate state. Biochemistry 42(29):8712-8719.
42. Johnson D T, et al (2020) Implementing In-Cell Fast Photochemical Oxidation of Proteins in a Platform Incubator with a Movable XY Stage. Anal Chem 92(2):1691-1696. PMCID:PMC7944481
43. Kaur P, et al (2015) Characterizing monoclonal antibody structure by carboxyl group footprinting. MAbs 7(3):540-552. PMCID:PMC4622658
44. Kaur P, et al (2015) Quantitative protein topography analysis and high-resolution structure prediction using hydroxyl radical labeling and tandem-ion mass spectrometry (MS). Mol Cell Proteomics 14(4):1159-1168. PMCID:PMC4390260
45. Kavungal D, et al. Artificial intelligence-coupled plasmonic infrared sensor for detection of structural protein biomarkers in neurodegenerative diseases. Sci Adv. 2023 Jul. 14; 9(28):eadg9644. doi: 10.1126/sciadv.adg9644. Epub 2023 Jul. 12.
46. Khaje N A, et al (2022) Validated determination of NRG1 Ig-like domain structure by mass spectrometry coupled with computational modeling. Commun Biol 5(1):452. PMCID:PMC9098640
47. Kitabatake N, et al (1988) Physicochemical and functional properties of hen ovalbumin dephosphorylated by acid phosphatase. Agricultural and Biological Chemistry 52(4):967-973.
48. Korir A K, et al (2008) Ultraperformance Ion-Pair Liquid Chromatography Coupled to Electrospray Time-of-Flight Mass Spectrometry for Compositional Profiling and Quantification of Heparin and Heparan Sulfate. Analytical Chemistry 80(4):1297-1306.
49. Krainer G, et al. Direct digital sensing of protein biomarkers in solution. Nat Commun. 2023 Feb. 6; 14(1):653. doi: 10.1038/s41467-023-35792-x.
50. Lees W J, et al (1994) Polyacrylamides bearing pendant alpha-sialoside groups strongly inhibit agglutination of erythrocytes by influenza A virus: multivalency and steric stabilization of particulate biological systems. Journal of medicinal chemistry 37(20):3419-3433.
51. Li C P, et al (2005) Phosphorylation of ovalbumin by dry-heating in the presence of pyrophosphate: effect on protein structure and some properties. J Agric Food Chem 53(12):4962-4967.
52. Li H, et al (2016) Time-Dependent Influence of Cell Membrane Permeability on MR Diffusion Measurements. Magn Reson Med 75(5):1927-1934. PMCID:PMC4676747.
53. Li, S. et al, Kinetics and mechanism studies of efficient degradation of 1-hexyl-3-methylimidazolium by zero-valent iron activated persulfate, Chemical Engineering Journal, Volume 460, 2023, 141575.
54. Li X, et al (2017) Structural Analysis of the Glycosylated Intact HIV-1 gp120-b12 Antibody Complex Using Hydroxyl Radical Protein Footprinting. Biochemistry 56(7):957-970. PMCID:PMC5319886
55. Li Z, et al (2015) High structural resolution hydroxyl radical protein footprinting reveals an extended Robo1-heparin binding interface. J Biol Chem 290(17):10729-10740. PMCID:PMC4409239
56. Liang Q, et al (2018) Improved de novo sequencing of heparin/heparan sulfate oligosaccharides by propionylation of sites of sulfation. Carbohydr Res 465:16-21. PMCID:PMC6286702
57. Lienqueo M E, et al (2007) Current insights on protein behaviour in hydrophobic interaction chromatography. J Chromatogr B Analyt Technol Biomed Life Sci 849(1-2):53-68.
58. Lindert S, et al (2013) Accelerated Molecular Dynamics Simulations with the AMOEBA Polarizable Force Field on Graphics Processing Units. J Chem Theory Comput 9(11):4684-4691. PMCID:PMC3948463
59. Liu H, et al (2019) Salt-free fractionation of complex isomeric mixtures of glycosaminoglycan oligosaccharides compatible with ESI-MS and microarray analysis. Sci Rep 9(1):16566. PMCID:PMC6851191
60. Liu H, et al (2020) Peracylation Coupled with Tandem Mass Spectrometry for Structural Sequencing of Sulfated Glycosaminoglycan Mixtures without Depolymerization. J Am Soc Mass Spectrom 31(10):2061-2072. PMCID:PMC7664153
61. Liu X R, et al (2019) Hydroxyl-Radical Reaction Pathways for the Fast Photochemical Oxidation of Proteins Platform As Revealed by (18)O Isotopic Labeling. Anal Chem 91(14):9238-9245. PMCID:PMC6635036
62. Luzete-Monteiro E et al (2022) Structures and consequences of pioneer factor binding to nucleosomes. Curr Opin Struct Biol 75:102425.
63. Matsumoto M L (2022) Molecular Mechanisms of Multimeric Assembly of IgM and IgA. Annual review of immunology 40:221-247.
64. McKenzie-Coe A A, et al (2021) Evaluating the Sulfate Radical Anion as a New Reagent for In-Cell Fast Photochemical Oxidation of Proteins. J Am Soc Mass Spectrom 32(7):1644-1647.
65. Miller J G et al (2001) Characteristics of xyloglucan after attack by hydroxyl radicals. Carbohydr Res 332(4):389-403.
66. Misra S K et al (2020) Enabling Real-Time Compensation in Fast Photochemical Oxidations of Proteins for the Determination of Protein Topography Changes. J Vis Exp (163). PMCID:PMC7695538
67. Misra S K, et al (2019) Compensated Hydroxyl Radical Protein Footprinting Measures Buffer and Excipient Effects on Conformation and Aggregation in an Adalimumab Biosimilar. AAPS J 21(5):87. PMCID:PMC7670911
68. Morgunova E et al (2021) Structural insights into the interaction between transcription factors and the nucleosome. Curr Opin Struct Biol 71:171-179.
69. Olson L J, et al (2020) Allosteric regulation of lysosomal enzyme recognition by the cation-independent mannose 6-phosphate receptor. Commun Biol 3(1):498. PMCID:PMC7481795
70. Pan X, et al (2020) Higher-Order Structure Influences the Kinetics of Diethylpyrocarbonate Covalent Labeling of Proteins. J Am Soc Mass Spectrom 31(3):658-665. PMCID:PMC7077735
71. Pan Y et al (2010) Membrane protein structural insights from chemical labeling and mass spectrometry. Analyst 135(6):1191-1200.
72. Pan Y, et al (2009) Mapping the structure of an integral membrane protein under semi-denaturing conditions by laser-induced oxidative labeling and mass spectrometry. J Mol Biol 394(5):968-981.
73. Pan Y, et al (2009) Structural characterization of an integral membrane protein in its natural lipid environment by oxidative methionine labeling and mass spectrometry. Anal Chem 81(1):28-35.
74. Pan Y, et al (2010) Site-directed mutagenesis combined with oxidative methionine labeling for probing structural transitions of a membrane protein by mass spectrometry. J Am Soc Mass Spectrom 21(11):1947-1956.
75. Pan Y, et al (2011) Kinetic folding mechanism of an integral membrane protein examined by pulsed oxidative labeling and mass spectrometry. J Mol Biol 410(1):146-158.
76. Pan Y, et al (2012) Validation of membrane protein topology models by oxidative labeling and mass spectrometry. J Am Soc Mass Spectrom 23(5):889-898.
77. Petitou M et al (2004) A synthetic antithrombin III binding pentasaccharide is now a drug! What comes next? Angewandte Chemie (International ed 43(24):3118-3133.
78. Pomin V H, et al (2010) Characterization of glycosaminoglycans by 15N NMR spectroscopy and in vivo isotopic labeling. Anal Chem 82(10):4078-4088. PMCID:2884402
79. Pomin V H, et al (2012) Exploiting enzyme specificities in digestions of chondroitin sulfates A and C: production of well-defined hexasaccharides. Glycobiology 22(6):826-838. PMCID:3336870
80. Reagan-Shaw S, et al (2008) Dose translation from animal to human studies revisited. FASEB J 22(3):659-661.
81. Ren, T.-L. et al, Degradation of imidazolium ionic liquids in a thermally activated ate system, Chemical Engineering Journal, Volume 412, 2021, 128624.
82. Rinas A, et al (2016) Development of a Microflow System for In-Cell Footprinting Coupled with Mass Spectrometry. Anal Chem 88(20):10052-10058.
83. Roush A E, et al (2020) Intrinsic Buffer Hydroxyl Radical Dosimetry Using Tris(hydroxymethyl)aminomethane. J Am Soc Mass Spectrom 31(2):169-172.
84. Saladino J, et al (2009) Aliphatic peptidyl hydroperoxides as a source of secondary oxidation in hydroxyl radical protein footprinting. J Am Soc Mass Spectrom 20(6):1123-1126.
85. Schafer H et al (2012) Tools for cellular immunology and vaccine research the in the guinea pig: monoclonal antibodies to cell surface antigens and cell lines. Vaccine 30(40):5804-5811.
86. Shami A A, et al. Dimethylthiourea as a Quencher in Hydroxyl Radical Protein Footprinting Experiments. J Am Soc Mass Spectrom. 2023 Dec. 6; 34(12):2864-2867. doi: 10.1021/jasms.3c00323. Epub 2023 Nov. 16.
87. Sharp J S, et al (2018) Real Time Normalization of Fast Photochemical Oxidation of Proteins Experiments by Inline Adenine Radical Dosimetry. Anal Chem 90(21):12625-12630. PMCID:PMC7811273
88. Sharp J S, et al (2021) Flash Oxidation (FOX) System: A Novel Laser-Free Fast Photochemical Oxidation Protein Footprinting Platform. J Am Soc Mass Spectrom 32(7):1601-1609.
89. Sheng L, et al (2019) Consequences of phosphorylation on the structural and foaming properties of ovalbumin under wet-heating conditions. Food Hydrocolloids 91:166-173.
90. Shortt R L, et al. Development of Spheroid-FPOP: An In-Cell Protein Footprinting Method for 3D Tumor Spheroids. J Am Soc Mass Spectrom. 2023 Mar. 1; 34(3):417-425. doi: 10.1021/jasms.2c00307. Epub 2023 Jan. 26.
91. Simon M D, et al (2007) The site-specific installation of methyl-lysine analogs into recombinant histones. Cell 128(5):1003-1012. PMCID:PMC2932701
92. Singh S, et al (2018) Monoclonal Antibodies: A Review. Curr Clin Pharmacol 13(2):85-99.
93. Stein P E, et al (1991) Crystal structure of uncleaved ovalbumin at 1.95 A resolution. J Mol Biol 221(3):941-959.
94. Tadi S et al (2021) Inline Liquid Chromatography-Fast Photochemical Oxidation of Proteins for Targeted Structural Analysis of Conformationally Heterogeneous Mixtures. Anal Chem 93(7):3510-3516.
95. Tandon R, et al (2021) Effective Inhibition of SARS-CoV-2 Entry by Heparin and Enoxaparin Derivatives. Journal of Virology 95.
96. Thiemann S et al (2016) Galectins and Immune Responses-Just How Do They Do Those Things They Do? Annual review of immunology 34:243-264.
97. Tong X, et al (2007) Effects of protein concentration on the extent of gamma-ray-mediated oxidative labeling studied by electrospray mass spectrometry. Anal Chem 79(16):6376-6382.
98. Tudella J, et al (2011) Oxidation of mannosyl oligosaccharides by hydroxyl radicals as assessed by electrospray mass spectrometry. Carbohydr Res 346(16):2603-2611.
99. Unger J, et al (2007) Measurements of free hemoglobin and hemolysis index: EDTA- or lithium-heparinate plasma? Clin Chem 53(9):1717-1718.
100. Vidarsson G, et al (2014) IgG subclasses and allotypes: from structure to effector functions. Front Immunol 5:520. PMCID:PMC4202688
101. Viola M, et al (2018) Subcutaneous delivery of monoclonal antibodies: How do we get there? J Control Release 286:301-314.
102. Vreeburg R A, A et al (2014) Fingerprinting of hydroxyl radical-attacked polysaccharides by N-isopropyl-2-aminoacridone labelling. Biochem J 463(2):225-237. PMCID:4170706
103. Wang K, et al (2021) Cryo-EM reveals the architecture of placental malaria VAR2CSA and provides molecular insight into chondroitin sulfate binding. (Research Square).
104. Wang X, et al (2011) Oligomeric Structure of the Chemokine CCL5/RANTES from NMR, MS, and SAXS Data. Structure 19(8):1138-1148.
105. Wang X, et al (2013) Chemokine oligomerization in cell signaling and migration. Progress in molecular biology and translational science 117:531-578. PMCID:3937849
106. Watson C et al (2012) Conformational analysis of therapeutic proteins by hydroxyl radical protein footprinting. AAPS J 14(2):206-217. PMCID:3326162
107. Weinberger S R, et al (2020) U.S. Pat. No. 10,816,468.
108. Weinberger S R, et al (2021) Laser-free Hydroxyl Radical Protein Footprinting to Perform Higher Order Structural Analysis of Proteins. J Vis Exp.
109. Wu A M et al (2019) Lectins and ELLSA as powerful tools for glycoconjugate recognition analyses. Glycoconjugate journal 36(2):175-183.
110. Xie B et al (2015) Hydroxyl Radical Dosimetry for High Flux Hydroxyl Radical Protein Footprinting Applications Using a Simple Optical Detection Method. Anal Chem 87(21):10719-10723. PMCID:PMC4631671
111. Xie B et al (2016) Relative Quantification of Sites of Peptide and Protein Modification Using Size Exclusion Chromatography Coupled with Electron Transfer Dissociation. J Am Soc Mass Spectrom 27(8):1322-1327. PMCID:PMC4945384
112. Xie B, et al (2017) Quantitative Protein Topography Measurements by High Resolution Hydroxyl Radical Protein Footprinting Enable Accurate Molecular Model Selection. Sci Rep 7(1):4552. PMCID:PMC5495787
113. Xiong S, et al (2008) Fractionation of proteins by heparin chromatography. Methods Mol Biol 424:213-221.
114. Xu G et al (2005) Radiolytic modification and reactivity of amino acid residues serving as structural probes for protein footprinting. Anal Chem 77(14):4549-4555.
115. Zhang B, et al (2018) Implementing fast photochemical oxidation of proteins (FPOP) as a footprinting approach to solve diverse problems in structural biology. Methods, San Diego, Calif 144:94-103.
116. Zhao J, et al (2018) Impact of Temperature on Heparin and Protein Interactions. Biochem Physiol 7(2). PMCID:PMC6156718
117. Ziemianowicz D S, et al (2017) Amino Acid Insertion Frequencies Arising from Photoproducts Generated Using Aliphatic Diazirines. J Am Soc Mass Spectrom 28(10):2011-2021.
118. Ziemianowicz D S, et al (2020) Correlation between Labeling Yield and Surface Accessibility in Covalent Labeling Mass Spectrometry. J Am Soc Mass Spectrom 31(2):207-216.
119. Zong C, et al (2016) Integrated Approach to Identify Heparan Sulfate Ligand Requirements of Robo1. J Am Chem Soc 138(39):13059-13067. PMCID:PMC5068570.

1. A method for evaluating a higher-order structure of a target biomolecule within a biological fluid, the method comprising:
(a) isolating a sample comprising a biological fluid from a multicellular host;
(b) subjecting the sample comprising the biological fluid to at least one labeling condition to produce a labeled target biomolecule;
(c) analyzing the labeled target biomolecule using targeted spectrometry to produce a labeled target molecule profile for the target biomolecule from a complex background; and
(d) comparing the labeled molecule profile to a reference molecule profile of an unmodified target biomolecule;
wherein a m/z (mass to charge) ratio and retention time of a labeled target biomolecule or degradation product thereof is modeled based on known chemical behavior of the at least one labeling condition; and
wherein a modeled retention time shift window is applied to a measured retention time of the unmodified target biomolecule or a degradation product thereof.

2. The method of claim 1, wherein the reference molecule profile comprises a labeling profile generated from the isolated, purified, or enriched target biomolecule from an in vitro sample, and wherein the reference molecule profile is used to model the labeled target molecule profile in the biological fluid in step (c).

3. The method of claim 1, wherein the biological fluid comprises whole blood, serum, plasma, urine, cerebrospinal fluid (CSF), or any combination thereof.

4. The method of claim 1, wherein the multicellular host is a mammal.

5. (canceled)

6. The method of claim 1, wherein the at least one labeling condition comprises an oxidizing condition, CF₃ radicals, SO₄ radicals, CO₃ radicals, I radicals, carbenes, aryl azides, benzophenones, or any combination thereof.

7. The method of claim 6, wherein subjecting the sample comprising the biological fluid to the oxidizing condition comprises contacting the biological fluid with at least one peroxide and irradiating the biological fluid and the at least one peroxide.

8. The method of claim 7, wherein the at least one peroxide comprises hydrogen peroxide (H₂O₂), sodium persulfate (Na₂S₂O₈), a mixed hydroperoxide, or any combination thereof.

9.-11. (canceled)

12. The method of claim 7, wherein the sample comprising the biological fluid and the at least one peroxide further comprises a radical scavenger.

13.-17. (canceled)

18. The method of claim 7, wherein the biological fluid and the at least one peroxide are irradiated with a UV lamp having a wavelength greater than or equal to about 200 nm.

19. (canceled)

20. The method of claim 1, further comprising quenching the sample comprising the biological fluid prior to performing step (c), wherein quenching comprises contacting the sample comprising the biological fluid with a quenching solution comprising an imidazole or quinone and a thioether.

21.-25. (canceled)

26. The method of claim 1, wherein the method further comprises contacting the sample comprising the biological fluid with a digestive enzyme, wherein the digestive enzyme comprises a protease, a nuclease, a lyase, a glycosidase, or any combination thereof.

27.-28. (canceled)

29. The method of claim 3, wherein the biological sample comprises whole blood, and wherein the method further comprises contacting the biological sample with a catalase inhibitor, a peroxidase inhibitor, or any combination thereof.

30. The method of claim 29, wherein the catalase inhibitor comprises hydroxylamine, 3-amino-1,2,4-triazole (3AT), aminoguanidine, thiosemicarbazide (TSC), or any combination thereof.

31. The method of claim 1, wherein the sample comprising the biological fluid is not purified prior to performing step (b) of the method.

32. The method of claim 1, wherein the target biomolecule comprises a protein, a peptide, a carbohydrate, a nucleic acid, or any combination thereof.

33.-37. (canceled)

38. A system for evaluating a higher-order structure of a target biomolecule within a biological fluid, the system comprising:
(a) a fluidic channel for receiving an isolated biological fluid comprising a target biomolecule;
(b) a mixing portion for admixing one or more labeling reagents with the isolated biological fluid to produce a labeled target biomolecule, wherein the mixing portion is coupled to the fluidic channel for receiving the isolated biological fluid; and
(c) an automated device for receiving the labeled target biomolecule from the mixing portion.

39. The system of claim 38, further comprising an irradiation module for irradiating the one or more labeling reagents and the isolated biological fluid.

40. The system of claim 39, further comprising a quenching module for quenching the one or more labeling reagents, a degradation product thereof, or both.

41. The system of claim 38, further comprising a second mixing portion for admixing one or more buffers with the isolated biological fluid.

42. The system of claim 38, further comprising an in-line spectrophotometry module for analysis of biological fluid integrity, the spectrophotometry module being located between the fluidic channel for receiving the isolated biological fluid and the mixing portion, wherein the in-line spectrophotometry module is configured to collect UV/vis spectra, fluorescence spectra, light scattering measurements, or any combination thereof.

43. (canceled)
